(19) United States
(12) Patent Application Publication
(10) Pub. No.: US 2023/0120179 A1
(43) Pub. Date: Apr. 20, 2023

(54) STORAGE MEDIUM, DISPLAY CONTROL APPARATUS AND DISPLAY SYSTEM

(71) Applicant: Konica Minolta, Inc., Tokyo (JP)

(72) Inventors: Kazuhiro ONO, Osaka (JP); Masaki SUNAGAWA, Osaka (JP)

(21) Appl. No.: 17/969,110

(22) Filed: Oct. 19, 2022

(30) Foreign Application Priority Data
Oct. 19, 2021 (JP) 2021-170589

(51) Int. Cl. G06F 16/955; G06F 3/0483; H04M 1/72445

(52) U.S. Cl. CPC G06F 16/955; G06F 3/0483; H04M 1/72445

(57) ABSTRACT

A non-transitory computer-readable storage medium stores a program for a computer of a display control apparatus that displays information in a first system and that is connected to a Web server of a second system over a network. The program causes the computer to: display patient information and examination information on a display region of a display screen displayed on a display of the first system, the display displaying information in the first system; obtain related information that is related to the patient information or the examination information from the Web server; and display the related information on the display region.

### CROSS-REFERENCE TO RELATED APPLICATIONS

The present invention claims priority under 35 U.S.C. § 119 to Japanese Patent Application No. 2021-170589, filed on Oct. 19, 2021, the entire contents of which being incorporated herein by reference.

### BACKGROUND

The present disclosure relates to a storage medium, a display control apparatus, and a display system.

### TECHNOLOGICAL FIELD

A system in a medical facility (e.g., radiology information system, dose control system, electronic medical record system, picture archiving and communication system (PACS), and image interpretation report system) can display, on its display, various kinds of information and medical images stored in other systems while displaying various kinds of information and medical images stored in its own system by executing an application.

According to JP2020-348115A, a first system sends out a uniform resource locator (URL) and a patient identification code to another system (second system) and allows a user to view reports stored in the second system by using a Web browser.

### SUMMARY

According to JP2020-348115A, the first system displays various kinds of information and medical images stored in the second system by using the Web browser while displaying various kinds of information and medical images on the display of the first system by executing the first system's application. In such a configuration, the first system needs to switch display contents between the first system's application screen and the Web browser screen. This may decrease the user's viewability and usability. Further, the Web browser screen may hide important information displayed on the first system's application screen (e.g., contraindication information of a user). This may incur the risk of overlooking the important information.

The present invention has been conceived in view of the above issues in the known art. Objects of the present invention include providing a storage medium, a display control apparatus, and a display system that can appropriately display information stored in the second system on the display of the first system.

To achieve at least one of the abovementioned objects, according to an aspect of the present invention, there is provided a non-transitory computer-readable storage medium storing a program for a computer of a display control apparatus that displays information in a first system and that is connected to a Web server of a second system over a network, the program causing the computer to: display patient information and examination information on a display region of a display screen displayed on a display of the first system, the display displaying information in the first system; obtain related information that is related to the patient information or the examination information from the Web server; and display the related information on the display region.

According to another aspect of the present invention, there is provided a display control apparatus that displays information in a first system and that is connected to a Web server of a second system over a network, the apparatus including a hardware processor that: displays patient information and examination information on a display region of a display screen displayed on a display of the first system, the display displaying information in the first system; obtains related information that is related to the patient information or the examination information from the Web server; and displays the related information on the display region.

According to another aspect of the present invention, there is provided a display system including: a Web server of a second system; and a display control apparatus that displays information in a first system and that is connected to the Web server over a network, wherein the display control apparatus includes a hardware processor that: displays patient information and examination information on a display region of a display screen displayed on a display that displays information in the first system; that obtains related information that is related to the patient information or the examination information from the Web server; and that displays the related information on the display region.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 shows an example of configuration of a display system in an embodiment of the present invention.

FIG. 2 shows an example of a board edit menu window.

FIG. 3 shows an example of the board edit menu window when “WORKLIST” is selected.

FIG. 4A shows an example of a board when “WORKLIST” is selected.

FIG. 4B shows an example of the board when “WORKLIST” is selected.

FIG. 5 is a flowchart of a Web browser display process.

FIG. 6 is a table showing examples of basic URLs.

FIG. 7 is a table showing examples of access URLs.

FIG. 8 is a flowchart of a user authentication process in a first modification.

FIG. 9 is a table showing examples of access URLs, authentication URLs, and authentication information in the first modification.

FIG. 10 shows an example of the board when “WORKLIST” is selected in a second modification.

### DETAILED DESCRIPTION OF THE EMBODIMENTS

An embodiment of a display system according to the present invention is described. However, the present invention is not limited to the illustrated examples.

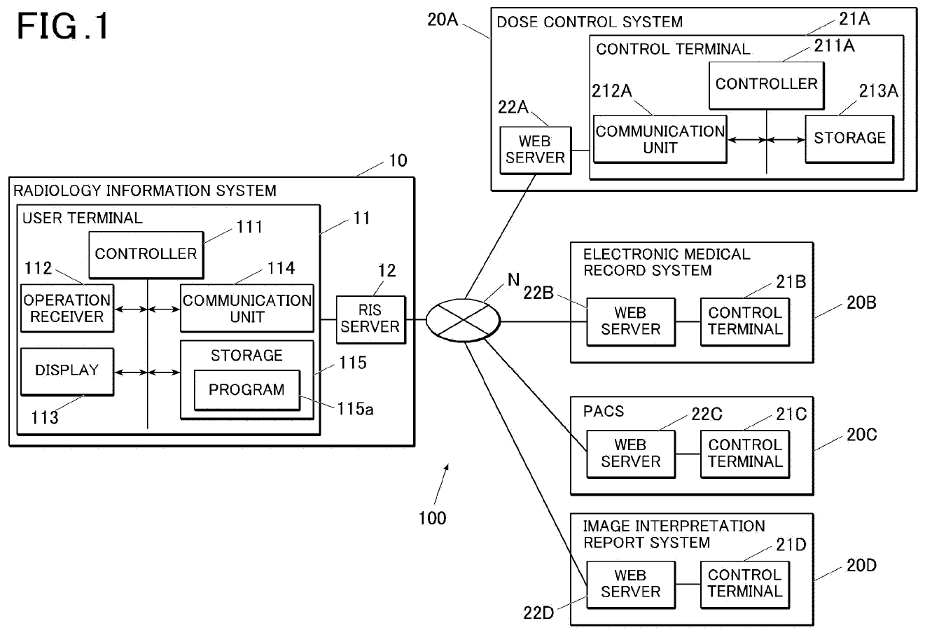

FIG. 1 shows an example of configuration of a display system 100 in an embodiment of the present invention.

As shown in FIG. 1, the display system 100 includes a radiology information system (RIS) 10 as a first system; and a dose control system 20A, an electronic medical record system 20B, a PACS 20C, and an image interpretation report system 20D as second systems. Hereinafter, each of the dose control system 20A, the electronic medical record system 20B, the PACS 20C, and the image interpretation report system 20D may be referred to as the second system 20 when they are not distinguished from each other. The number of second systems 20 is not limited to a specific number.

The RIS 10 and the second systems 20 are connected so as to send and receive data to and from each other via a network N, such as a local area network (LAN) built in a medical facility.

The RIS 10 includes a user terminal 11 as the display control apparatus and an RIS server 12.

The user terminal 11 is connected to the RIS server 12. The RIS server 12 is a computer device that displays, on a display 113 (described later), various kinds of information and medical images accumulated and stored in the RIS server 12 or a Web server(s) 22 (described later).

The user terminal 11 includes a controller 111 (hardware processor), an operation receiver 112, the display 113, a communication unit 114, and a storage 115. These components are connected via a bus.

The controller 111 includes a central processing unit (CPU) and a random access memory (RAM) and centrally controls operations of the components of the user terminal 11. Specifically, the controller 111 reads out a system program or various processing programs stored in the storage 115, loads them into a work area in the RAM, and performs various processes in accordance with the programs.

The controller 111 displays patient information and examination information on a display region of a display screen displayed on the display 113, which displays information in the RIS 10 (first system). The controller 111 thus functions as a display control unit.

The controller 111 also obtains related information (described later) related to the patient information or the examination information from the Web server 22. The controller 111 thus functions as an obtaining unit.

The controller 111 stores (saves), in the RIS server 12, at least part of the related information obtained from the Web page data stored in the Web server 22. The at least part of the related information is stored as the patient information or the examination information. The controller 111 thus functions as a storing unit.

The operation receiver 112 includes a keyboard including cursor keys, character and number input keys and various function keys, and a pointing device, such as a mouse. The operation receiver 112 outputs, to the controller 111, a command signal input by a key operation on the keyboard or by a mouse operation. The operation receiver 112 receives various operations by the user.

The display 113 consists of a monitor, such as a liquid crystal Display (LCD), and displays various screens/windows, medical images, and the display screen for the display application (described later) in accordance with commands of display signals input by the controller 111.

The communication unit 114 is an interface that sends and receives data to and from external devices (e.g., the second systems 20) via the RIS server 12. The communication unit 114 sends and receives data to and from devices connected to the network N.

The storage 115 consists of a hard disk and/or a nonvolatile semiconductor memory on which data and programs can be written and from which data and programs can be read out. The storage 115 stores programs 115a.

The programs 115a include various programs to be executed by the controller 111, such as system programs, application programs, and a display application program for displaying various kinds of information and medical images.

The storage 115 stores basic URLs to be described later.

The RIS server 12 is a computer device connected to the network N. The RIS server 12 accumulates, stores and manages various kinds of information and medical image data.

Specifically, the RIS server 12 stores patient information. The patient information includes basic patient information, disability information, and contraindication information.

The basic patient information is information on each patient. The basic patient information includes the patient ID in the medical facility, patient name, age, date of birth, sex, blood type, height, and weight, for example.

The disability information is information on a physical disability/disorder of the patient. The disability information includes the state of the physical disability (e.g., hearing impairment, disturbance of consciousness).

The contraindication information is information on a contraindication(s) of the patient (e.g., the patient's susceptibility to allergic reactions).

As the patient information, the RIS server 12 also stores patient comments, patient comments per radiologist, patient comments per examination, and HIS (hospital information system) patient comments.

The RIS server 12 stores examination information (e.g., examination number, examined part, examination direction, examination method, examination posture, right/left).

As the examination information, the RIS server 12 also stores an examination list, an examination timeline, comments at examination, and a message.

Each of the second systems 20 includes a control terminal 21 and a Web server 22.

The control terminal 21 includes a controller 211, a communication unit 212, and a storage 213. These components are connected via a bus.

The controller 211 includes a CPU and a RAM and centrally controls processing operations of the components of the control terminal 21. Specifically, the controller 211 reads out a system program or various processing programs stored in the storage 213, loads them into a work area in the RAM, and performs various processes in accordance with the programs.

The communication unit 212 is an interface that sends and receives data to and from external devices (e.g., the RIS 10) via the Web server 22. The communication unit 212 sends and receives data to and from devices connected to the network N.

The storage 213 consists of a hard disk and/or a nonvolatile semiconductor memory on which data and programs can be written and from which data and programs can be read out.

The Web server 22 is a computer device connected to the network N. The Web server 22 accumulates, stores and manages various kinds of information and medical image data.

The Web server 22 stores a Web server program and so forth. The Web server program causes the Web server 22 to function as a Web server that provides various Web pages to the display region of the display screen that is displayed by the display application installed in the user terminal 11.

The Web server 22 stores data of Web pages having access URLs each of which is associated with patient information or examination information. The Web page shows various kinds of information and medical images. For example, the data of a Web page is hypertext markup language (HTML) data for displaying various kinds of information and medical images.

Specifically, in a case where the second system 20 is the dose control system 20A, the various kinds of information and medical images are supplementary information (e.g., patient ID, patient name, date of birth, age, sex, height, and weight); examination information (examination number, examination date and time, medical image generation device, type of imaging, examined part, imaging technique, and radiopharmaceutical); and medical images that are generated by the medical image generation device that is stored in association with dose information.

In a case where the second system 20 is the electronic medical record system 20B, the various kinds of information and the medical images are diagnosis record data (information on the patient's conditions and diagnosis results) and prescription data.

In a case where the second system 20 is the PACS 20C, the various kinds of information and the medical images are medical images that are generated in the medical facility that is stored in association with the patient information and/or the examination information. The medical images are digital medical images digitally generated by the medical image generation device, such as a computed radiography (CR) device, a computed tomography (CT) device, and a magnetic resonance imaging (MRI) device.

In a case where the second system 20 is the image interpretation report system 20D, the various kinds of information and the medical images are medical images and management information. The management information includes multiple pieces of information, such as the examination number, the patient ID of the subject, patient name, sex, date of birth, examination date, examination time, examination conditions (e.g., imaged part, imaging direction, posture), examination description, type of modality, state of image interpretation (e.g., not interpreted yet, under interpretation, pending approval, approved), unique ID (UID) of medical images obtained in the examination, place where the file of medical images is stored, report ID of the image interpretation report of the examination, place where the interpretation report is stored, ID of the image interpretation doctor, and ID of the approving doctor. The ID of the image interpretation doctor is the user ID of the doctor who created the image interpretation report. The ID of the approving doctor is the user ID of the doctor who approved the image interpretation report.

Next, the display application to be activated in the user terminal 11 is described.

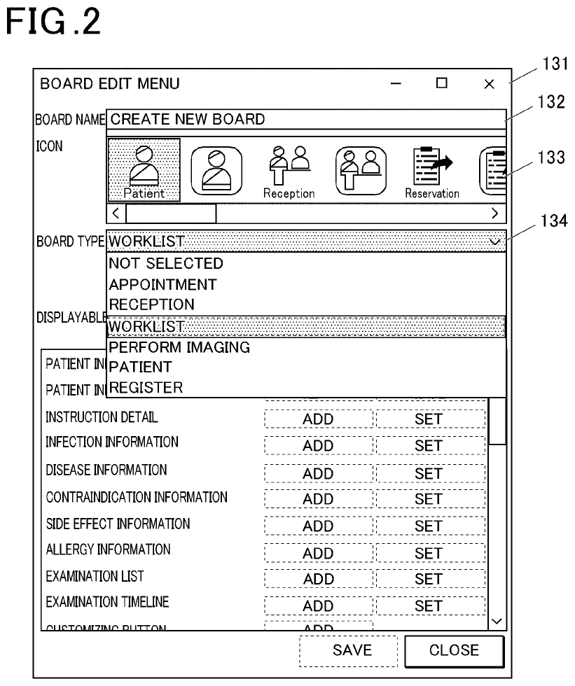

FIG. 2 shows a board edit menu window 131 for editing the display region (board) of the display screen that is displayed by the display application.

In the section 132 in FIG. 2, the user can set the board name as desired through inputs with the operation receiver 112. In the example shown in FIG. 2, the board name is set to “create new board”.

In the selection part 133 shown in FIG. 2, the user can select an icon to be added to the board as desired. In the example shown in FIG. 2, the icon of “Patient” is selected.

In the selection part 134 shown in FIG. 2, the user can select a board type. For the respective board types, items that can be displayed on the board (displayable items) are set beforehand based on uses. For example, in order to set the board on the display screen as the board for examination appointments, “APPOINTMENT” is selected. In order to set the board on the display screen as the board for processing receptions, “RECEPTION” is selected. In order to set the board on the display screen as the board for an examination list, “WORKLIST” is selected. In order to set the board on the display screen as the board for the time of performing imaging, “PERFORM IMAGING” is selected. In order to set the board on the display screen as the board for registering and checking patient information, “PATIENT” is selected. In order to set the board on the display screen as the board that shows the worklist as a register in which the schedule of the worklist is shown, “REGISTER” is selected. In the example shown in FIG. 2, “WORKLIST” is selected.

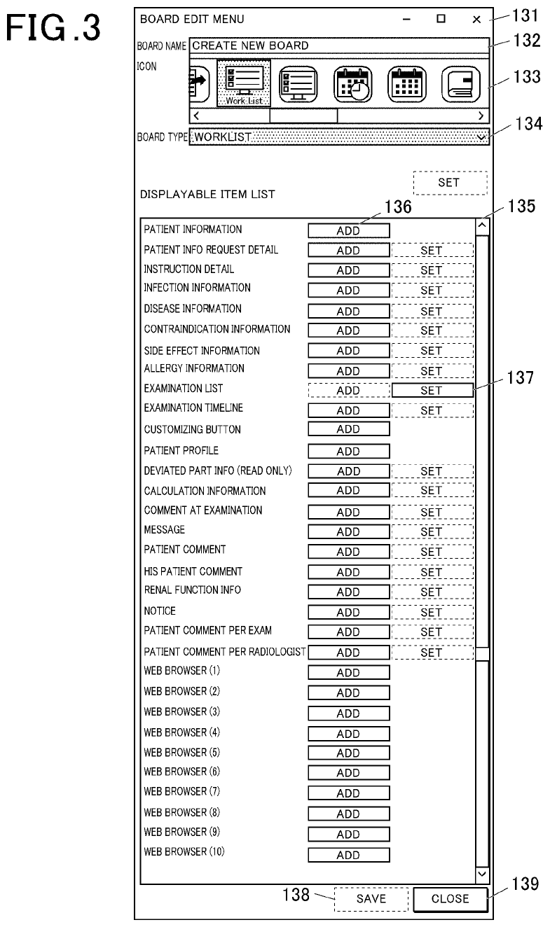

FIG. 3 shows the board edit menu window 131 when “WORKLIST” is selected in the selection part 134.

In the selection part 135 in FIG. 3, the user can select an item(s) to be displayed on the board by clicking an “ADD” button 136 from among the displayable items that are set beforehand for the “WORKLIST” type board. In the “WORKLIST” type board, the “examination list” is a necessary item and is set beforehand to be always displayed. The user can do various settings on the “examination list” item by clicking the “SET” button 137.

The user can save and set the information selected in the board edit menu window 131 in the storage 115 by clicking the “SAVE” button 138. The user can close the board edit menu window 131 by clicking the “CLOSE” button 139. When the board edit menu window 131 is closed, a board corresponding to the saved settings opens.

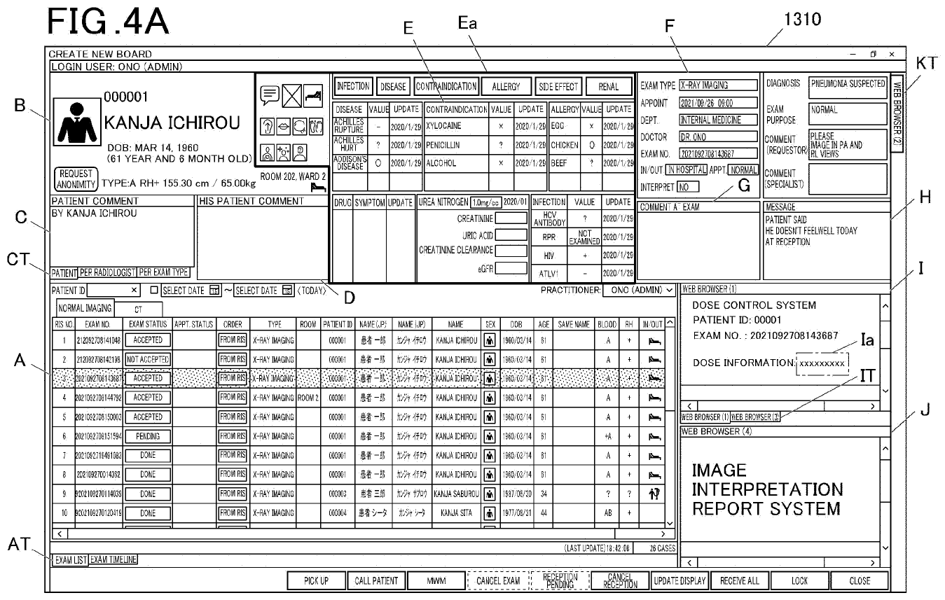

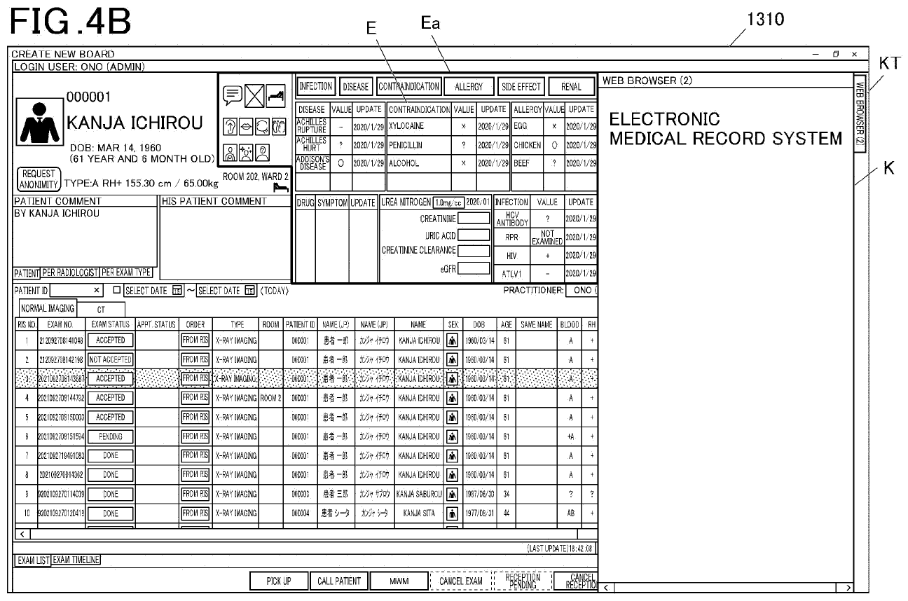

FIGS. 4A, FIG. 4B show an example of the board 1310 when “WORKLIST” is selected.

The controller 111 displays, on the board 1310, the items selected in the selection part 135 in FIG. 3 and the examination list.

The controller 111 obtains the examination list and the examination timeline from the RIS server 12 and displays either the examination list or the examination timeline in the region A. Herein, the user selects either the examination list or the examination timeline by selecting a tab AT with the operation receiver 112. The user selects an examination that the user wants to view from among the examinations registered in the examination list by manipulating the operation receiver 112. In the example shown in FIGS. 4A, FIG. 4B, the examination with the RIS number of 3 is selected.

When the user selects an examination from among the examinations registered in the examination list, the controller 111 obtains the basic patient information related to the selected examination from the RIS server 12 and displays the obtained basic patient information in the region B.

The controller 111 also obtains patient comments, patient comments per radiologist, and patient comments per examination type that are related to the selected examination from the RIS server 12. The controller 111 then displays one among the obtained patient comments, patient comments per radiologist, and patient comments per examination type in the region C. Herein, the user selects one among the obtained patient comments, patient comments per radiologist, and patient comments per examination type by selecting a tab CT with the operation receiver 112.

The controller 111 also obtains HIS patient comments related to the selected examination from the RIS server 12 and displays the obtained HIS patient comments in the region D.

The controller 111 also obtains the contraindication information related to the selected examination from the RIS server 12 and displays the obtained contraindication information in the region E.

The controller 111 also obtains the examination information related to the selected examination from the RIS server 12 and displays the obtained examination information in the region F.

The controller 111 also obtains comments at examination related to the selected examination from the RIS server 12 and displays the obtained comments at examination in the region G.

The controller 111 also obtains a message related to the selected examination from the RIS server 12 and displays the obtained message in the region H.

The controller 111 displays either the Web browser (1) or the Web browser (3) in the region I. Herein, the user selects either the Web browser (1) or the Web browser (3) by selecting a tab IT with the operation receiver 112.

The controller 111 displays the Web browser (4) in the region J.

When the user positions the mouse cursor on the tab KT by manipulating the operation receiver 112, the controller 111 displays the web browser (2) in the region K, as shown in FIG. 4B.

The contents displayed in the Web browsers (1) to (4) are described later.

Thus, the display contents regarding specific items can be switched with tabs. This allows the display region of the display screen to maintain the amount of information displayed thereon even when the Web browser screen is displayed in the display region of the display screen.

As shown in FIGS. 4A, FIG. 4B, the Web browser screen is displayed in a region different from regions in which the examination list and the items except the Web browser selected in the selection part 135 in FIG. 3 are displayed. That is, the controller 111 displays (i) the first display region in which the patient information and the examination information are displayed and (ii) the second display region in which the related information is displayed, the related information being displayed on the Web browser screen, such that the first display region and the second display region are separate from each other in the display region. In FIGS. 4A, FIG. 4B, the first display region includes the regions A, B, C, D, Ea (described later), F, G, and H, whereas the second display region includes the regions I, J, and K.

Thus, the region Ea that shows important information including contraindication information is not hidden behind the Web browser screens (Web browsers (1) to (4)).

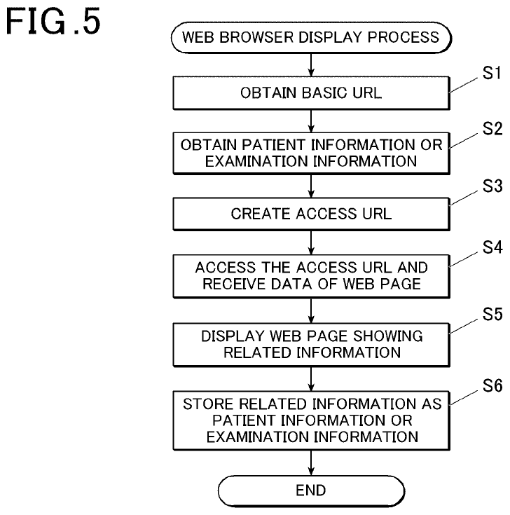

When the user selects a Web browser (Web browsers (1) to (4)) as an item to be displayed on the board 1310 as shown in FIGS. 4A, FIG. 4B, the controller 111 of the user terminal 11 performs the Web browser display process shown in FIG. 5. By the process, the controller 111 displays, on the Web browser screen, various kinds of information and medical images stored in the Web server 22.

In the Web browser display process, the controller 111 firstly obtains from the storage 115, the basic URL corresponding to the Web browser selected in the selection part 135 in FIG. 3 (Step S1). The basic URL is determined beforehand for each of the Web browsers.

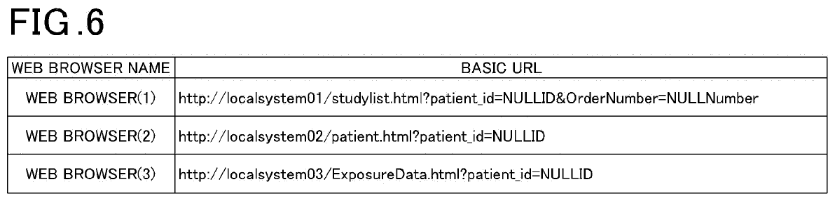

FIG. 6 shows examples of basic URLs of the Web browsers (1) to (3).

The controller 111 then obtains, from the RIS server 12, patient information or examination information related to the examination selected from the examination list by the user (Step S2). The patient information is the patient ID in the basic patient information, for example. The examination information is the examination number, for example.

The controller 111 replaces a predetermined character string in the basic URL with the patient information or examination information obtained in Step S2 to generate an access URL (Step S3). Specifically, the controller 111 replaces the character string “NULLID” in the basic URL shown in FIG. 6 with the patient ID obtained in Step S2. Further, the controller 112 replaces the character string “NULLNumber” in the basic URL with the examination number obtained in Step S2.

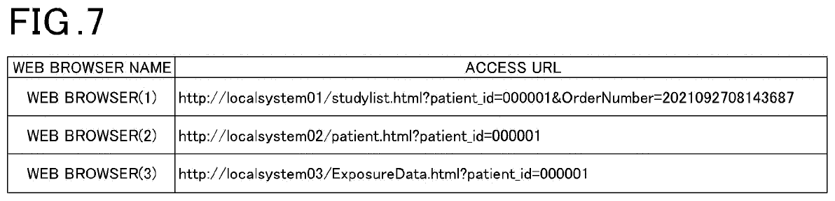

FIG. 7 shows examples of access URLs of the Web browsers (1) to (3) generated in Step S3. In the examples shown in FIG. 7, the patient ID is “000001”, and the examination number is “2021092708143687”. That is, the access URL is associated with the patient ID as the patient information and/or the examination number as the examination information.

The controller 111 accesses the access URL generated in Step S3 via the communication unit 114. The controller 111 then receives data of the Web page that is given the generated access URL and that is stored in the Web server 22 (Step S4). The data of the web page is HTML data for displaying various kinds of information and medical images, for example. That is, the various kinds of information and medical images to be displayed as the data of the Web page, which is stored in the Web server 22, is the related information related to the patient information or the examination information.

On the basis of the data of the Web page received in Step S4, the controller 111 displays the Web page on the Web browser screen in the board in the display screen (Step S5). That is, the controller 111 displays, on the display region of the display screen, the Web page that shows the related information by using the Web browser.

In the example shown in FIG. 4A, the Web browser (1) displayed in the region I shows the Web page stored in the dose control system 20A, wherein the Web page shows dose information as the related information. The Web browser (4) displayed in the region J shows the Web page stored in the image interpretation report system 20D. In the example shown in FIG. 4B, the Web browser (2) displayed in the region K shows the Web page stored in the electronic medical record system 20B. The illustration of the related information shown in the Web browsers (2) and (4) is omitted.

On the basis of the data of the Web page received in Step S4, the controller 111 stores, in the RIS server 12, a desired piece(s) of related information in the related information displayed on the Web page as the patient information or the examination information (Step S6) and ends the process. Specifically, when the controller 111 receives HTML data for showing various kinds of information in Step S4 for example, the controller 111 obtains text data from a predetermined part of the HTML page and stores the obtained text data in the RIS server 12 as the patient information or examination information. The predetermined part from which the information is obtained is determined beforehand for each of the Web browsers.

In the example shown in FIG. 4A, the predetermined part from which the information is obtained is the region Ia in the region I. In the case, the controller 111 stores, in the RIS server 12, the value of dose information shown in the region Ia as the patient information or examination information.

By the above Web browser display process, images of the patient obtained in the past and stored in the Web server 22 can be displayed on the display region of the display screen at the time of examinations, for example. When the second system 20 is the dose control system 20A, the dose information stored in the Web server 22 can be displayed on the display region of the display screen.

Further, the information displayed on the display region of the display screen can be stored in the RIS server 12.

[First Modification]

Next, a first modification of the above embodiment is described.

Hereinafter, aspects of the first modification different from the above embodiment are mainly described. The display system 100 in the first modification is the same as the display system 100 in the above embodiment.

In the first modification, the controller 111 of the user terminal 11 associates an authentication URL (described later) for user authentication with authentication information (described later) necessary for user authentication and stores and sets the associated authentication URL and the authentication information in the storage 115. The controller 111 thus functions as a first setting unit.

The controller 111 performs user authentication by using the authentication URL and the authentication information before obtaining the data of the Web page stored in the Web server 22. The controller 111 thus functions as an authentication unit.

The storage 213 of the control terminal 21 in the second system 20 stores an authentication information list in which authentication information of users who are permitted to view the Web page are registered.

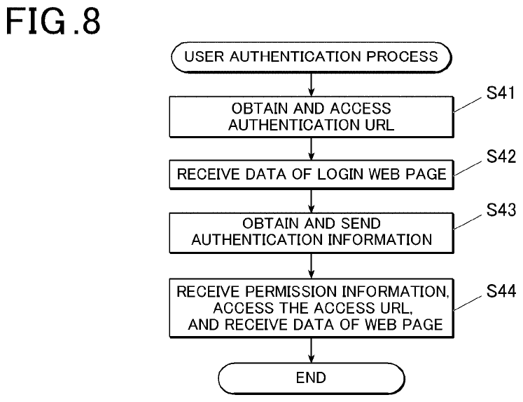

In Step S4 of the Web browser display process in the first modification, the controller 111 performs the user authentication process shown in FIG. 8.

In the user authentication process, the controller 111 firstly obtains from the storage 115, the authentication URL corresponding to the Web browser selected in the selection part 135 in FIG. 3 and accesses the authentication URL (Step S41). The authentication URL is determined beforehand for each of the Web browsers.

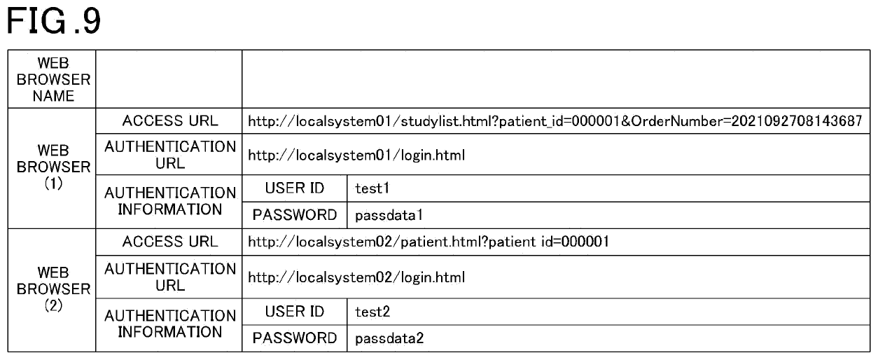

FIG. 9 shows examples of authentication URLs of the Web browsers (1) and (2).

The controller 111 receives data of a login Web page from the second system 20 (Step S42).

The controller 111 obtains from the storage 115, the authentication information corresponding to the Web browser selected in the selection part 135 in FIG. 3 and sends the authentication information to the second system 20 (Step S43). The authentication information is associated with the authentication URL and set beforehand by the controller 111.

FIG. 9 shows examples of authentication information for the Web browsers (1) and (2). The authentication information is a user ID and a password, for example.

In response to receiving the authentication information from the RIS 10, the controller 211 of the control terminal 21 refers to the authentication information list stored in the storage 213 and judges whether the received authentication information is registered in the authentication information list. When judging that the received authentication information is registered in the authentication information list, the controller 211 sends permission information to the RIS 10. The permission information is to permit viewing the Web page.

In response to receiving the permission information from the second system 20, the controller 111 accesses the access URL generated in Step S3. The controller 111 receives data of the Web page that is given the generated access URL and that is stored in the Web server 22 (Step S44). The controller 111 then ends the user authentication process and proceeds to Step S5 of the Web browser display process.

In the user authentication process, the authentication information is not included in the authentication URL or the access URL. This guarantees the security. Further, the authentication information is automatically sent by the controller 111. This allows the user to omit manually inputting the authentication information.

In the user authentication process, Step S42 may be omitted.

[Second Modification]

Next, a second modification of the above embodiment is described.

Hereinafter, aspects of the second modification different from the above embodiment are mainly described. The display system 100 in the second modification is the same as the display system 100 in the above embodiment.

In the second modification, the controller 111 of the user terminal 11 associates a specific key operation (described later) with a specific process on the Web page stored in the Web server 22, and stores and sets the associated key operation and process as shortcut information in the storage 115. The controller 111 thus functions as a second setting unit.

As the specific process on the Web page, the controller 111 outputs at least either the patient information or the examination information to the Web page. The controller 111 thus functions as a shortcut unit.

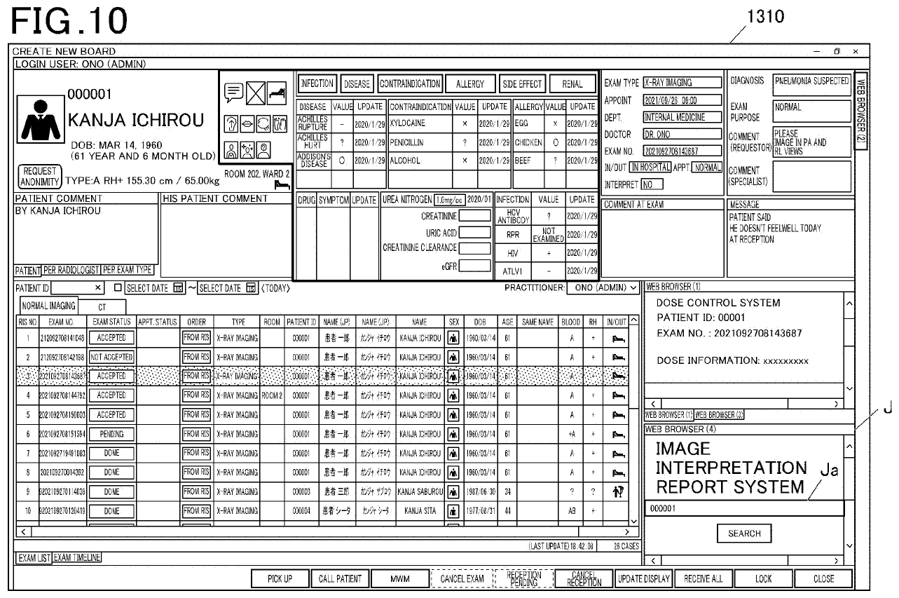

Herein, the basis URL is not set for the Web browser (4) displayed in the region J in FIG. 10.

Assume that a Web browser for which the basic URL is not determined is displayed on the board 1310. When a specific key operation is made with the operation receiver 112 by the user, the controller 111 of the user terminal 11 obtains from the storage 115, shortcut information corresponding to the specific key operation. On the basis of the obtained shortcut information, the controller 111 obtains, from the RIS server 12, at least either the patient information or examination information related to the examination selected in the examination list. The controller 111 then outputs the obtained information to the Web page shown by the Web browser for which the basic URL is not determined.

The specific key operation is an operation of pressing a shortcut key (e.g., the F1 key of the keyboard of the operation receiver 112 is pressed) or a mouse gesture by the user, for example.

The shortcut information indicates a process that is performed when the user presses a shortcut key (e.g., F1 key), for example. For example, in the process, the patient ID related to the examination selected in the examination list is output to the input section Ja of the Web browser (4) shown in FIG. 10, for which the basic URL is not determined. In the example shown in FIG. 10, the patient ID “000001” that is related to the examination selected in the examination list is output to the input section Ja of the Web browser (4).

In the shortcut information, the specific key operation and the specific process on the Web page that is performed by the controller 111 in response to the specific key operation can be determined as desired.

According to the above configuration, the user can search for information stored in the Web server 22 and narrow down the search on the basis of the patient information and/or the examination information stored in the RIS server 12.

Even when the basic URL is not set for a Web browser, the controller 111 can use the patient information/examination information stored in the RIS server 12 to display the Web page corresponding to the patient information/examination information.

As described above, according to the embodiments, the display system 100 includes the Web server 22 of the second system 20 and the display control apparatus (the user terminal 11) that displays information in the first system (the RIS 10) and that is connected to the Web server 22 over the network N. The user terminal 11 includes the controller 111 (display control unit, obtaining unit) that displays patient information and examination information on the display region (the board 1310) of the display screen displayed on the display 113 that displays information in the first system; that obtains related information that is related to the patient information or the examination information from the Web server 22; and that displays the related information on the display region (the board 1310).

According to such a configuration, the controller 111 can appropriately display, on the display region of the display screen displayed on the display 113 of the first system, the related information related to the patient information, or the examination information stored in the second system 20.

Further, according to the above embodiments, in the display system 100, the controller 111 (obtaining unit, storing unit) obtains the related information from data of the Web page stored in the Web server 22 and stores at least part of the related information as the patient information or the examination information, the related information being obtained from the data of the Web page.

According to such a configuration, the first system can easily store the related information that is related to the patient information or the examination information and that is stored in the second system 20.

Further, according to the above embodiments, in the display system 100, the second system 20 may be an electronic medical record system, a PACS, an image interpretation report system, or a dose control system.

According to such a configuration, the first system can appropriately display the related information that is related to the patient information or the examination information and that is stored in the electronic medical record system, the PACS, the image interpretation report system, or the dose control system.

Further, according to the above embodiments, in the display system 100, the controller 111 (display control unit) displays the related information on the display region by using a Web browser.

According to such a configuration, the first system can easily display the related information that is related to the patient information or the examination information and that is stored in the second system 20 by using the Web browser.

Further, according to the above embodiments, in the display system 100, the controller 111 (display control unit) displays the first display region and the second display region separately in the display region, the first display region displaying the patient information and the examination information, the second display region displaying the related information.

According to such a configuration, the patient information and the examination information are not hidden behind the Web browser screen. Therefore, the display 113 of the first system can appropriately display the related information that is related to the patient information or the examination information and that is stored in the second system 20.

Further, according to the above embodiments, in the display system 100, the patient information includes at least one of basic patient information, disability information, and contraindication information.

According to such a configuration, important information such as disability information and contraindication information included in the patient information are not hidden behind the Web browser screen. Therefore, the display 113 of the first system can appropriately display the related information that is related to the patient information or the examination information and that is stored in the second system 20.

Further, according to the above embodiments, in the display system 100, the controller 111 (first setting unit, authentication unit) sets an authentication URL for user authentication in association with authentication information required for the user authentication and performs the user authentication by using the authentication URL and the authentication information before obtaining data of the Web page stored in the Web server 22.

According to such a configuration, the authentication information is not included in the authentication URL or the access URL for accessing the Web page. This guarantees the security. Further, the authentication information is automatically sent by the controller 111. This allows the user to omit manually inputting the authentication information.

Further, according to the above embodiments, in the display system 100, the controller 111 (second setting unit) sets a specific key operation in association with a specific process on the Web page stored in the Web server 22.

According to such a configuration, the information stored in the Web server 22 can be displayed by a simple operation.

Further, according to the above embodiments, in the display system 100, the controller 111 (shortcut unit) outputs at least either the patient information or the examination information onto the Web page as the specific process on the Web page.

According to such a configuration, the user can search for information stored in the Web server 22 and narrow down the search on the basis of the patient information and/or the examination information.

The embodiments described above are examples of the present invention and not intended to limit the present invention. The detailed configuration and detailed operation of the components constituting the system can be appropriately modified without departing from the scope of the present invention.

For example, although the controller 111 replaces a predetermined character string in the basic URL with a patient ID or an examination number in Step S3 of the Web browser display process in the above embodiment, the present invention is not limited to this. The predetermined character string in the basic URL may be replaced with information on an examined part as the examination information.

According to such a configuration, images of the respective examined parts that have been taken by a normal imaging method and stored in the Web server 22 can be displayed on the display region of the display screen at the time of examinations, for example.

In the selection part 134 shown in FIG. 2 of the above embodiments, when “PERFORM IMAGING” is selected for the board for the time of performing imaging, the patient information and/or the examination information to be displayed on the board are determined at the time the board is opened. Therefore, the controller 111 obtains the patient information and/or the examination information to be displayed on the board from the RIS server 12 in Step S2 of the Web browser display process.

In the display system 100 of the above embodiments, the first system is the RIS 10. However, the present invention is not limited to this. The first system may be any one of the dose control systems 20A, the electronic medical record system 20B, the PACS 20C, and the image interpretation report system 20D. In the case, the RIS is one of the second systems 20.

Further, the first and second systems are not limited to medical systems such as an RIS, dose control system, electronic medical record system, PACS, and image interpretation report system. The first and second systems may be systems different from the medical systems.

The program for executing various processes in the respective apparatuses may be stored in a portable storage medium(s). Further, as a medium to provide data of the programs via a communication line, a carrier wave can be used.

Although embodiments of the present invention have been described and illustrated in detail, the disclosed embodiments are made for purposes of illustration and example only and not limitation. The scope of the present invention should be interpreted by terms of the appended claims.

What is claimed is:

1. A non-transitory computer-readable storage medium storing a program for a computer of a display control apparatus that displays information in a first system and that is connected to a Web server of a second system over a network, the program causing the computer to:
display patient information and examination information on a display region of a display screen displayed on a display, the display displaying information in the first system;
obtain related information that is related to the patient information or the examination information from the Web server; and
display the related information on the display region.

2. The storage medium according to claim 1, wherein the program causes the computer to:
obtain the related information from data of a Web page stored in the Web server; and
store at least part of the related information as the patient information or the examination information, the related information being obtained from the data of the Web page.

3. The storage medium according to claim 1, wherein the second system is an electronic medical record system, a picture archiving and communication system, an image interpretation report system, or a dose control system.

4. The storage medium according to claim 1, wherein the program causes the computer to display the related information on the display region by using a Web browser.

5. The storage medium according to claim 1, wherein the program causes the computer to display a first display region and a second display region separately in the display region, the first display region displaying the patient information and the examination information, the second display region displaying the related information.

6. The storage medium according to claim 5, wherein the patient information includes at least one of basic patient information, disability information, and contraindication information.

7. The storage medium according to claim 1, wherein the program causes the computer to:
set an authentication URL for user authentication in association with authentication information required for the user authentication; and
perform the user authentication by using the authentication URL and the authentication information before obtaining data of a Web page stored in the Web server.

8. The storage medium according to claim 1, wherein the program causes the computer to set a specific key operation in association with a specific process on a Web page stored in the Web server.

9. The storage medium according to claim 8, wherein the program causes the computer to output at least either the patient information or the examination information onto the Web page as the specific process on the Web page.

10. A display control apparatus that displays information in a first system and that is connected to a Web server of a second system over a network, the apparatus comprising a hardware processor that:
displays patient information and examination information on a display region of a display screen displayed on a display, the display displaying information in the first system;
obtains related information that is related to the patient information or the examination information from the Web server; and
displays the related information on the display region.

11. A display system comprising:
a Web server of a second system; and
a display control apparatus that displays information in a first system and that is connected to the Web server over a network, wherein the display control apparatus includes a hardware processor that:
displays patient information and examination information on a display region of a display screen displayed on a display, the display displaying information in the first system;
obtains related information that is related to the patient information or the examination information from the Web server; and
displays the related information on the display region.
